(19) United States
(12) Patent Application Publication
(10) Pub. No.: US 2025/0152756 A1
(43) Pub. Date: May 15, 2025

(54) SURFACE MODIFICATION OF MICRO-BUBBLES AND NANOPARTICLES BASED ON POLY(ALKYLCYANOACRYLATE)

(71) Applicant: RHEINISCH-WESTFÄLISCHE TECHNISCHE HOCHSCHULE (RWTH) AACHEN, Aachen (DE)

(72) Inventors: Fabian Kiessling, Aachen (DE); Twan Lammers, Maaseik (BE); Yang Shi, Aachen (DE); Junlin Chen, Aachen (DE)

(21) Appl. No.: 18/836,238

(22) PCT Filed: Feb. 2, 2023
(86) PCT No.: PCT/EP2023/052500

§ 371 Date: Aug. 6, 2024

(30) Foreign Application Priority Data
Feb. 7, 2022 (DE) 10 2022 102 770.8

(51) Int. Cl. A61K 49/22; A61K 47/62

(52) U.S. Cl. CPC A61K 49/227; A61K 47/62

(57) ABSTRACT

The present invention relates, inter alia, to a method for functionalizing a cyanoacrylate-based material comprising the steps of providing a cyanoacrylate-based material having ester groups; providing a ligand having an amino group; contacting the cyanoacrylate-based material with the ligand under conditions favoring aminolysis of the amino group of the ligand and the ester groups of the cyanoacrylate-based material; and optionally obtaining the cyanoacrylate-based material functionalized with the ligand.

### TECHNICAL FIELD

The invention lies in the field of functionalization of cyanoacrylate-based material, in particular butyl cyanoacrylate-based material. More specifically, the present invention relates to a method for coupling a compound to cyanoacrylate-based material by means of aminolysis and its use in the production or functionalization of ultrasound (US) contrast enhancers and US-mediated drug delivery systems.

### BACKGROUND TO THE INVENTION

Ultrasound (US) is one of the most frequently used diagnostic methods. It is a non-invasive, comparatively inexpensive imaging method with a wide range of applications. In combination with microbubbles (MBs), US allows functional and molecular imaging of (patho-) physiological phenomena. MBs are gas-filled vesicles (bubbles) of 1 to 5 μm in size whose shell is stabilized by lipids, proteins or polymers. Contrast enhancement is caused by a compressible gas core that enables the bubble to reflect the applied US waves. Loaded with drugs, the MBs can also act as a US-mediated drug delivery system that can be disrupted by US pulses in vivo to release the drug locally.

In particular, poly(butyl cyanoacrylate) (PBCA) MBs, an MB variant with a rather hard shell, is considered a suitable candidate for diagnostics and therapy. PBCA is a biodegradable polymer and is approved by the FDA as a surgical superglue for wound closure. The shell of PBCA MB usually consists of relatively small polymer chains with an average molecular weight (MG) of 4 kDa, with more than 99% of the chains below 40 kDa. The diameter of PBCA MBs is typically around 2 μm, and the thickness of the shell can vary between 50 and 300 nm. This relatively thick shell enables stable encapsulation of the gas and prevents its diffusion out of the MBs.

The coupling of functional compounds to the shell of MBs can expand the field of application of US MBs. Accordingly, since the first PBCA-MBs were developed by emulsion polymerization, multi-step syntheses have been used to couple antibodies and peptides in order to produce targeted PBCA MBs for molecular US imaging.

A common method for the functionalization of MBs is biotin-streptavidin conjugation. Palmowski et al. (Invest Radiol. 2008 March; 43 (3): 162-9) produced streptavidin-coated PBCA MBs and investigated their physicochemical properties. Subsequently, several studies demonstrated that these MBs can be functionalized by conjugation with monoclonal antibodies that recognize receptor proteins involved in various diseases.

A disadvantage of the biotin-streptavidin conjugation is that the MBs produced are only of limited clinical use in humans due to the potential immunogenicity of streptavidin. In addition, the biotin-streptavidin conjugation is a non-covalent bond, which means that there is a risk of the functional compound detaching from the MBs. The lower binding strength also makes it more difficult to bind larger substances, e.g. nanoparticles and micelles. In addition, biotin-streptavidin conjugation is a rather slow process, which can lead to low yields of intact MBs due to the rather short lifetime and instability of the MBs. The relatively high costs and the complexity of the synthesis also stand in the way of widespread application.

Another modification strategy is based on 1-ethyl-3-(3-dimethylaminopropyl) carbodiimide (EDC), which is able to activate carboxyl groups of PBCA MBs in aqueous solution for the coupling of amines to amide bonds. The challenges with this modification strategy are control and reproducibility of the reaction. The technique requires hydrolysis to access carboxyl groups, which enables further coupling with EDC. The degree of hydrolysis is difficult to control. Usually a pH of 10 to 11 is required. The excess of hydroxyl groups enhances the hydrophilic properties of PBCA and reduces the hydrophobic interactions in the MBs, resulting in destruction of the structure and formation of short PBCA chains. Experiments showed that the number of MBs was reduced by around 48% after hydrolysis and EDC coupling.

The primary problem of the present invention was therefore to provide a method for modifying or functionalizing cyanoacrylate-based MBs which can be used in particular as US contrast enhancers or US-mediated drug delivery systems. Furthermore, the process should be as efficient, controllable, inexpensive and/or simple as possible. Furthermore, it was desired that the MBs produced have a high functionalization density and are immunologically safe for humans.

According to the invention, the problem is at least partially solved in that a method for functionalizing a cyanoacrylate-based material comprises the following steps:
a) preparation of a cyanoacrylate-based material with ester groups;
b) provision of a ligand with an amino group;
c) contacting the cyanoacrylate-based material and the ligand under conditions favoring aminolysis of the amino group of the ligand and the ester groups of the cyanoacrylate-based material; and optionally:
d) obtaining the cyanoacrylate-based material functionalized with the ligand.

The present invention is based on the finding that aminolysis enables the covalent coupling of a ligand with an amino group in a particularly efficient, reproducible, simple and cost-effective manner and is capable of producing a functionalized material that has a number of advantageous properties in addition to functionalization. Compared to the biotin-streptavidin conjugation chemistry, the coupling efficiency is many times higher. Furthermore, the reaction is easier to control so that a constant coupling efficiency can be achieved. The coupling is gentler; the yield of intact material is higher. The process is simple and cost-effective. The material produced in this way has good acoustic properties (additionally, reference is made to the example section).

A further advantage is that the material produced according to the invention is basically immunologically safe. As already indicated above, there are concerns regarding the immunogenicity of streptavidin present on known prior art MBs, since it is of non-human origin and has a high molecular weight. The method according to the invention thus represents a form of coupling that is harmless in vivo or at least less questionable. Due to the low immunogenicity, a longer in vivo circulation time can also be expected.

The present method is not based on the use of amphiphilic shell material as known, for example, from DE 198 82 362 T5. This type of microparticle has a shell consisting of two layers. The outer layer exposed to blood and tissue consists of an amphiphilic biocompatible material such as polyethylene glycol, polyethylene oxide, polypropylene glycol and combinations or derivatives thereof. The inner layer consists of a biodegradable polymer which is intended to provide the shell with the desired mechanical and acoustic properties or properties for the transportation of drugs. An emulsification process is suitable for the production of the microparticles. Due to the amphiphilicity of the material forming the outer layer, stable oil/water emulsions can be produced. The shell can be stabilized by cross-linking using glutaraldehyde or carbodiimide. As already mentioned, the cyanoacrylate-based material according to the invention is not an amphiphilic material. For example, the monomer n-butyl cyanoacrylate (PubChem ID: 23087) has a log P value of 2.4 and is therefore hydrophobic. The polymer PBCA based on this monomer can therefore only be more hydrophobic and not hydrophilic. Consequently, the material according to the invention is not produced by conventional emulsification and/or emulsion polymerization.

The advantages described can be exploited particularly well when the method according to the invention is used for the preparation of functionalized poly(alkyl cyanoacrylate) (PACA) MBs to be used as a functionalized US contrast enhancer or as a functionalized US-mediated drug delivery system. The ligand to be coupled is preferably a compound that functionalizes the produced PACA MBs in such a way that they accumulate in certain areas of the body, thus enabling or at least supporting target-oriented diagnostics or target-oriented therapy.

Further aspects, embodiments and advantages of the invention become apparent from the detailed description and the experimental part together with the drawings and the claims.

### BRIEF DESCRIPTION OF THE DRAWING

FIG. 1 shows an HPLC chromatogram at 220 nm wavelength of cRGD-modified PBCA MBs. Unmodified PBCA MBs showed no absorbance peak due to the lack of a corresponding chromogenic group. cRGD-modified PBCA MBs had a different retention time compared to free cRGD, indicating the formation of cRGD-modified PBCA MBs.

FIG. 2 shows the particle size distribution of PBCA MBs modified according to the invention at pH 8 (“aminolysis”) in comparison to unmodified PBCA MBs (“control”) and in comparison to PBCA MBs modified at pH 10 via EDC (“hydrolysis”).

FIG. 3 shows a comparison of the targeting efficiency of cRGD-modified PBCA MBs (FIG. 3C, RGD MBs) to TNF-alpha activated endothelial cells from human umbilical vein endothelial cells (HUVEC) compared to cRAD-modified PBCA MBs (FIG. 3B, RAD MBs) and unmodified PBCA MBs (FIG. 3A, control) using in vitro flow chamber assays (100 x, scale bar 50 μm). The MBs were loaded with rhodamine B. Hoechst and WGA 488 were used to stain the nuclei and cell membrane, respectively. The arrows show the red staining.

FIG. 4 shows the echogenicity of cRGD-modified PBCA MBs (FIG. 4A bottom, FIG. 4C) in comparison to the control (FIG. 4A top, FIG. 4B) before (FIG. 4A, left) and after (FIG. 4A, right) bursting. The time of bursting is indicated in FIG. 4B and FIG. 4C. In the US imaging, no contrast enhancement was measurable after bursting (black image, see FIG. 4A, right), indicating that the contrast signal measured before bursting originated from the (still intact) MBs.

FIG. 5 shows an example of how modified MBs can be produced by modifying PBCA MBs with an antibody as a ligand.

### DETAILED DESCRIPTION

In a first aspect, the invention relates to a method for functionalizing a cyanoacrylate-based material (also referred to herein as “material” for short). The method according to the invention comprises the following steps:
a) preparation of a cyanoacrylate-based material with ester groups;
b) provision of a ligand with terminal amino groups;
c) contacting the cyanoacrylate-based material and the ligand under conditions favoring aminolysis between the amino groups and the ester groups; and optionally:
d) obtaining the cyanoacrylate-based material functionalized with the ligand (also referred to herein as “functionalized material” for short).

The term “cyanoacrylate” or “alkyl cyanoacrylate” refers to an alkyl ester of cyanoacrylic acid. A polymeric shell composed of alkyl cyanoacrylates accordingly comprises polyalkyl cyanoacrylates (also abbreviated herein as “PACA”). Polyalkyl cyanoacrylates refer to polymers consisting of one or more alkyl cyanoacrylates that are essentially free of free acid and alcohol groups. The term “cyanoacrylate-based material” refers to a material that contains cyanoacrylate in unpolymerized, partially polymerized or polymerized form. Optionally, the material may contain other components, in particular other polymers that are not based on cyanoacrylates. In a preferred embodiment of the invention, the cyanoacrylate-based material is at least partly present as PACA, further preferably as PACA with an alkyl group having 2 to 10 C atoms, preferably 3 to 9 C atoms, more preferably 4 to 8 C atoms. A particularly preferred PACA in the context of the present invention is PBCA and is known, for example, from EP 3 223 864. PBCA is referred to chemically exactly as poly(n-butyl cyanoacrylate).

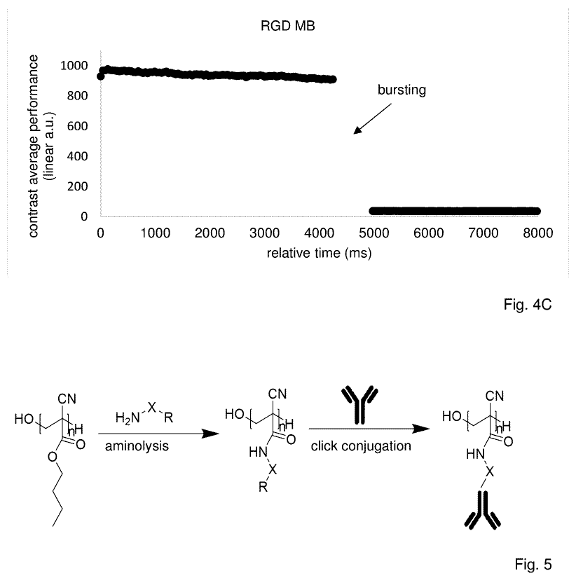

If the material in step c) is unpolymerized, the modification is carried out on the ester groups of the cyanoacrylate monomers. After modification, polymerization can optionally take place, so that modified material based on PACA, such as PACA MBs, in particular PBCA MBs, can also be obtained in this way. Preferably, the material is already polymerized in step c) and the (accessible) ester groups of the polymer are modified (see also FIG. 5).

In a preferred embodiment of the invention, the amino group is a primary amino group (NH₂) which is bound directly to the ligand R (H₂N—R) or is bound to the ligand R via oxygen (H₂NO—R). Aminolysis according to the following reaction equation is particularly preferred (the cleaved 1-butanol is not shown)

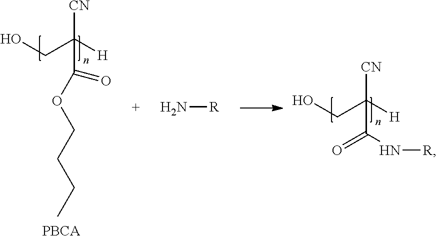

wherein —R represents the ligand or —X—R′ in which X represents oxygen and R′ represents the ligand.

Regardless of whether the term “ligand” is used in the singular or plural, it refers collectively to a group of identical molecules to be coupled. The same applies to the following term “functional compound”. However, the term “a ligand” does not exclude the possibility that the cyanoacrylate-based material is functionalized with more than one ligand, i.e., for example, has a first molecular group and a second molecular group different from the first molecular group.

Preferred methods within the scope of the present invention use a functional compound as a ligand. A functional compound refers to a compound that imparts a desired function to the material modified thereby. Preferably, the functional compound is selected from the group consisting of targeting ligands, diagnostics, therapeutics, macromolecules and nanomedicine constructs.

Targeting ligands are compounds that have an affinity for a predetermined target site, such as a specific tissue type, and in this way effect or at least support the transport of the corresponding functionalized material to a desired target site. Preferred target ligands are peptides for integrin-mediated cell adhesion or non-peptide analogs thereof, or antibodies directed against specific target structures. RGD peptides are particularly preferred. The term “RGD peptide” refers to a peptide with an amino acid sequence consisting of the three amino acids arginine, glycine and aspartic acid, RGD for short. The RGD sequence occurs naturally in the extracellular matrix (EM). This enables cells to bind to the EM with the help of integrins. This property can be utilized in the context of the present invention by presenting RGD peptides (or non-peptide analogs) on the surface of the cyanoacrylate-based material. This is to achieve that the so functionalized material accumulates in integrin-rich tissues, such as cytokine-activated blood vessels (e.g. damaged arteries), tumors and their blood vessels and inflammatory intestinal segments. Another preferred target ligand is E-selectin, a cell adhesion molecule expressed by endothelial cells activated by cytokines such as TNFα or IL-1. Antibodies that bind integrins, selectins and/or cell adhesion molecules are further examples of preferred target ligands.

Although it is preferred that—in the case of use as a drug carrier described below-a drug (active ingredient) is passively incorporated into the polymer shell, the coupling process can also be used to conjugate active ingredients.

A preferred ligand with a therapeutic effect is doxorubicin, an anthracycline used in chemotherapy (cytostatic drug).

Macromolecules to be coupled include, in particular, proteins and other polymers such as RNA or DNA, especially mRNA and plasmid DNA, which impart desired (surface) properties to the cyanoacrylate-based material. For example, polymers such as polyethylene glycol (PEG) can be coupled to increase the in vivo circulation time. Modifying the surface with a hydrophilic compound such as PEG can (also) minimize the interaction of the active substance on the surface with the blood vessel cells. This can, for example, prevent the active substance from exerting its (possibly here undesired) effect on the way to the target site.

The term “nanomedicine constructs” refers to nanoparticles with an average particle diameter of 1 nm to 999 nm that have therapeutically or diagnostically relevant properties, such as polymer micelles and liposomes.

Most preferred ligands are the already mentioned peptides for integrin-mediated cell adhesion or non-peptide analogs thereof, in particular RGD peptides such as RGDfK. RGD peptides are preferably coupled to PBCA MBs for specific binding and to aid in the diagnosis of inflammatory bowel disease, atherosclerosis, arterial wall injury, cancer or other disease associated with vascular inflammation.

Before using the functionalized material, in particular the functionalized MBs, characterization can optionally be carried out. Exemplary characterization methods are described in the example section. A specific test method is the determination of the targeting efficiency of RGD-functionalized material in comparison to a RAD-functionalized material (negative control).

In an alternative embodiment, the ligand is a binding member (so-called linker), which is first bound (in step c)) to the cyanoacrylate-based material. A functional compound (as defined above) can then be bound via the linker. For this purpose, in addition to the amino group, preferably the primary amino group, the linker has one or more further groups which can be coupled to the desired functional compound. Alternatively, the ligand to be coupled in step c) may comprise a functional compound and a linker which are already linked to each other.

PEGs or alkanes with a preferably terminal coupling group opposite the amino group are particularly suitable as linkers. Preferred coupling groups are selected from the group consisting of azides, alkynes and maleimides. The structure of specific linkers can be described as follows:

X—R₁—Y, wherein
R₁ is —(CH)₂ₙ— with n=1, 2, 3 or more, or —(CH₂—CH₂—O)ₙ— with n=1, 2, 3 or more,
X is —NH₂ or —O—NH₂, and
Y is azide, alkyne or maleimide.

After coupling the linker as a ligand to the cyanoacrylate-based material within the context of the aminolysis (step c)), the functional compound R₂ can be bound to the linker, for example by one of the following catalyzed click reactions:
Huisgen dipolar cycloaddition:

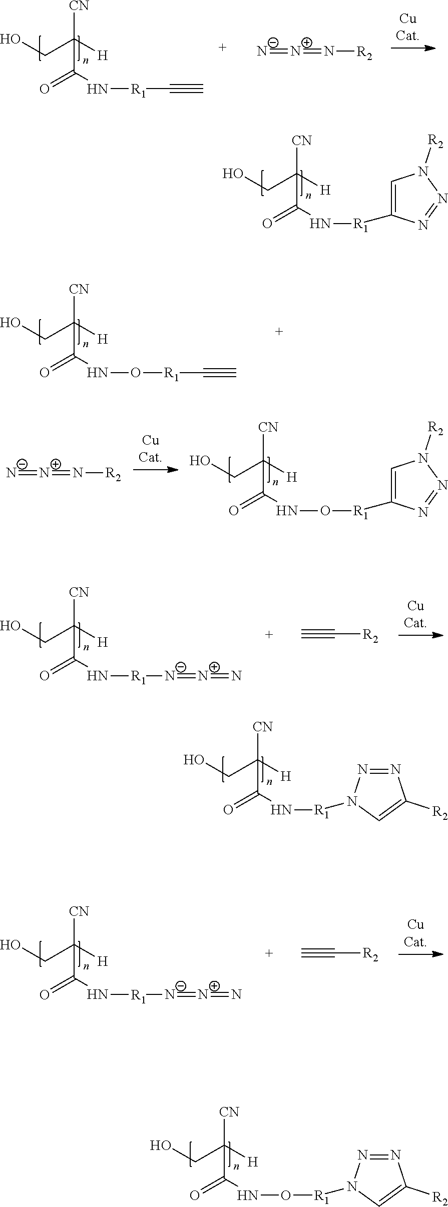

Michael addition:

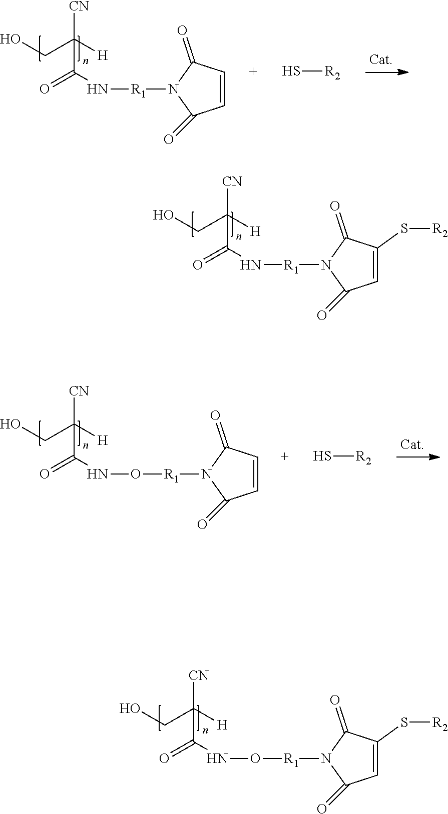

In a preferred embodiment, the cyanoacrylate-based material is already provided in step c) in polymerized form, preferably in the form of particles, capsules or vesicles whose mean diameter is preferably in the nano (1 nm to 999 nm) and/or micrometer range (1 μm to 999 μm). The particles, capsules or vesicles also preferably have an average diameter of 500 nm to 8 μm. Deviating from the meaning of the terms nano and micrometer range, vesicles with an average diameter of 0.1 μm to 100 μm are referred to as MBs.

A particularly preferred embodiment concerns the use of the functionalized material as a US contrast enhancer. For this purpose, the cyanoacrylate-based material is provided in the form of vesicles, in particular MBs, and subjected to aminolysis. The vesicles have a polymer shell and a gas core enclosed by the polymer shell, the gas core preferably containing air, oxygen, nitrogen oxide and/or perfluorocarbon. In principle, it is also possible, and encompassed by the present invention, that the gas core is only formed from a liquid core as a result of excitation with ultrasound. The wall thickness is preferably in the range from 10 to 400 nm, in particular 50 to 300 nm. The average molecular weight is preferably 1 kDa to 20 kDa, in particular 2 kDa to 10 kDa, with preferably more than 90%, in particular more than 95% of the chains being below 50 kDa, in particular below 40 kDa. Alternatively, it is possible to first functionalize the cyanoacrylate monomers and then form vesicles. The production of vesicles from cyanoacrylate monomers is well known to those skilled in the art.

A further, also preferred embodiment relates to use as a US-mediated drug delivery system. For this purpose, the gas-filled vesicles also have a drug that can be embedded in the matrix or the lumen and/or bound to the surface of the polymer shell, for example. For example, the drug can be bound to PACA via physicochemical interactions. Ultrasonic treatment can be used to control the release of the drug from the drug delivery system. The intensity and wavelength of the ultrasound source, which is usually located outside the body, is selected so that the radiation passes through the tissue to the desired release site. US pulses are used to burst the vesicles to release the drug. Drug delivery systems which, when exposed to ultrasound, release a drug embedded therein are known to the skilled person under the English term “ultrasound-mediated drug delivery”.

Instead of coupling another compound such as a target ligand to the ester group, it is generally possible for the drug to be the ligand. A targeted release of the drug from the drug delivery system can be controlled by selecting the body region to be treated with ultrasound. However, it is preferable that the surface of the drug delivery system is functionalized in such a way that it binds specifically to certain in vivo structures, as described above.

Step c) contacting the cyanoacrylate-based material and the ligand under conditions which favor aminolysis between the amino groups of the ligand and the ester groups of the cyanoacrylate-based material is preferably carried out in aqueous, in particular buffered, solution, preferably at a pH of 7.25 to 8.75, more preferably 7.5 to 8.5, most preferably 7.75 to 8.25. Alternatively or additionally, step c) may be carried out in the presence of a catalyst, in particular a lithium catalyst such as lithium methanolate.

It is also preferable to carry out the aminolysis in the presence of a surfactant in order to prevent or at least reduce aggregation of the particles, capsules or vesicles. Triton X-100, which can be added to the reaction mixture in a final concentration of 0.02%, is mentioned here as an example.

Another aspect of the present invention relates to a cyanoacrylate-based material functionalized with a ligand, produced according to the method of the invention. The features and embodiments described in connection with the method according to the invention form corresponding features and embodiments of the functionalized material produced thereby. This applies accordingly to the other aspects of the invention described herein.

A US contrast enhancer and a US-mediated drug delivery system comprising the aforementioned functionalized cyanoacrylate-based material represent further aspects of the present invention. Here, the functionalized material is in the form of gas-filled vesicles, in particular MBs. As described above, desired properties can be imparted on the vesicles by the process according to the invention. In the case of the US contrast enhancer according to the invention and the US-mediated drug delivery system according to the invention, functionalization with target ligands are particularly envisaged in order to achieve enrichment at the desired target site.

As examples, which equally represent further aspects of the invention, the use of the contrast agent or drug delivery system according to the invention in the context of diseases associated with vascular inflammation, in particular inflammatory bowel disease, arteriosclerosis, arterial wall injury or cancer, may be mentioned. Thus, the US contrast enhancer according to the invention is particularly suitable for their detection in the context of a diagnosis, and the US-mediated drug delivery system according to the invention is particularly suitable for their treatment. In particular, it is envisaged that the contrast agent is functionalized with one or more ligands which specifically bind to one or more tissue markers which are indicative of the presence of the disease in order to be able to determine the expression of the tissue marker(s) in a tissue by means of US imaging. For use as a drug delivery system, it is particularly envisaged that the drug delivery system is functionalized with one or more ligands that specifically bind to one or more tissue markers that are indicative of the presence of the disease in order to specifically enrich the drug delivery system in a tissue in which the expression of the tissue marker(s) is high. Accordingly, the use of the contrast enhancer according to the invention for detecting, diagnosing or supporting a diagnosis of a disease associated with vascular inflammation, in particular inflammatory bowel disease, arteriosclerosis, arterial wall injury or cancer, and a corresponding diagnostic method represent further aspects of the invention. For this purpose, the ultrasound contrast enhancer comprises vesicles functionalized with a peptide for integrin-mediated cell adhesion with a polymer shell and a gas core enclosed by the polymer shell, produced according to the method of the invention.

Further aspects relate to the drug delivery system according to the invention for use in the treatment of a disease associated with vascular inflammation, in particular inflammatory bowel disease, arteriosclerosis, arterial wall injury or cancer, and a corresponding method of treatment. Here, it is envisaged that the ultrasound-mediated drug delivery system comprises vesicles functionalized with a peptide for integrin-mediated cell adhesion, having a polymer shell and a gas core enclosed by the polymer shell, produced according to the method of the invention, and a drug. A preferred drug is the above-mentioned doxorubicin.

Another aspect of the invention relates to a composition comprising the US contrast enhancer or US-mediated drug delivery system of the invention and a dispersant in which the US contrast enhancer or US-mediated drug delivery system is present, or a lyophilizate thereof. Suitable dispersants include sodium chloride, PBS (Phosphate Buffered Saline), HBSS (Hanks' Balanced Salt Solution) and other physiological buffers and solutions.

The present invention will now be described further with reference to the following examples and the accompanying drawings.

### Examples

Using functionalization of PBCA MBs with cRGD (Cyclo(Arg-Gly-Asp-D-Phe-Lys)) as an example, the known technology described in the introduction (hereinafter also referred to as the streptavidin process or EDC process) were compared with the method according to the invention based on the aminolysis described below.

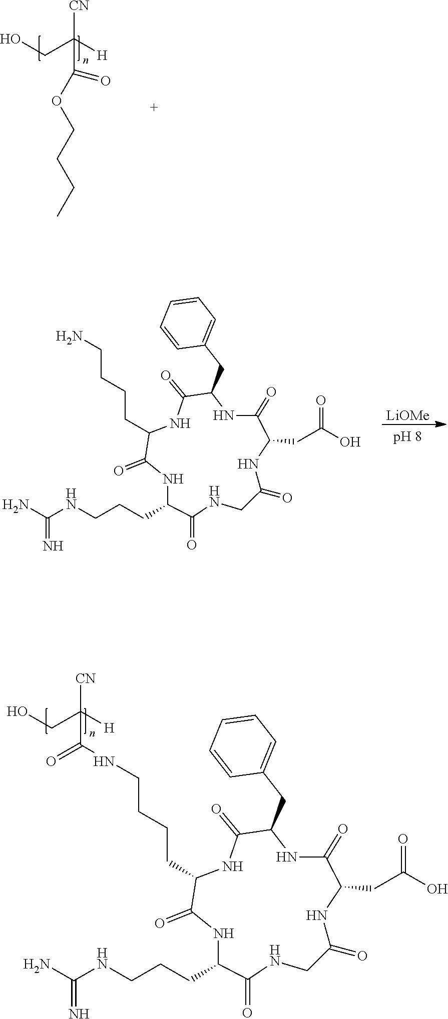

The results are as follows:

### 1. Efficiency of the Modification

According to previous studies, quantification of the number of coupled streptavidin molecules on the surface of PBCA MBs using FACS revealed an average of 95 streptavidin proteins/MB (equivalent to 15.7×10⁻²³ mol/MB). This means that no more than 95 binding sites are available for functionalization. In comparison, a coupling efficiency of 3.84×10⁻¹⁶ mol/MB (according to Pierce's quantitative colorimetric peptide assay) was achieved with the method according to the invention using the coupling of cRGD to PBCA MBs as an example.

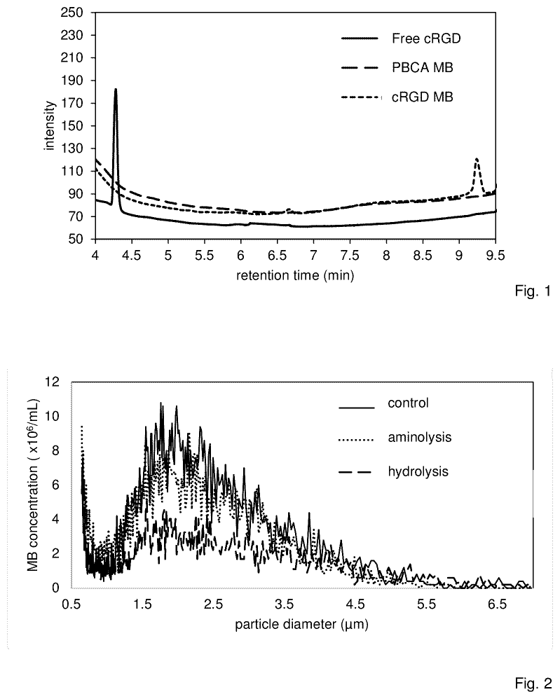

Furthermore, a coupling efficiency of 10.53±1.5% was achieved (see FIG. 1). This means that a good 10% cRGD was coupled to the PBCA MBs.

### 2. Controllability

The current biotin-streptavidin conjugation chemistry for the modification of PBCA MBs has the disadvantage that the coupling efficiency is difficult to control. Even under the same coupling conditions, the average efficiency varies depending on the synthesis method and streptavidin coating protocol (e.g. degree of MB hydrolysis prior to streptavidin coating). The method according to the invention is well controllable. In particular, a consistent coupling efficiency can be achieved, whereby material with consistent properties can be produced (data not shown).

### 3. Yield

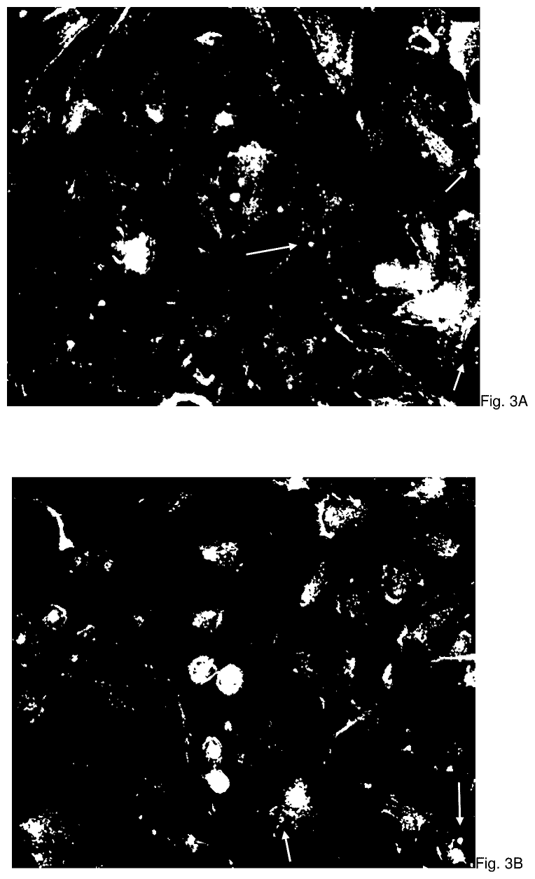

Compared to the EDC process, the yield of the method according to the invention is higher. As already mentioned, the known technique requires hydrolysis to make carboxyl groups accessible, which enables further coupling with EDC. The degree of hydrolysis is difficult to control. Usually a pH of 10 to 11 is required. The excess of hydroxyl groups can trigger rapid depolymerization of the PCA MBs, which destroys the structure and generates short PBCA chains. Thus, experiments showed that the number of MBs was reduced by about 48% after hydrolysis and EDC coupling. In contrast, the method according to the invention can largely prevent the degradation of PCA MBs. Using the coupling of cRGD to PBCA MBs as an example, a loss of only about 10% of the MBs was observed (see FIG. 2).

### 4. Costs and Complexity

As already described in the introduction, the streptavidin process is relatively expensive, complicated and time-consuming (multi-stage synthesis, long incubation times). The process according to the invention, on the other hand, is inexpensive and can be carried out in a simple one-pot step.

### 5. Properties of the Functionalized PBCA MBs

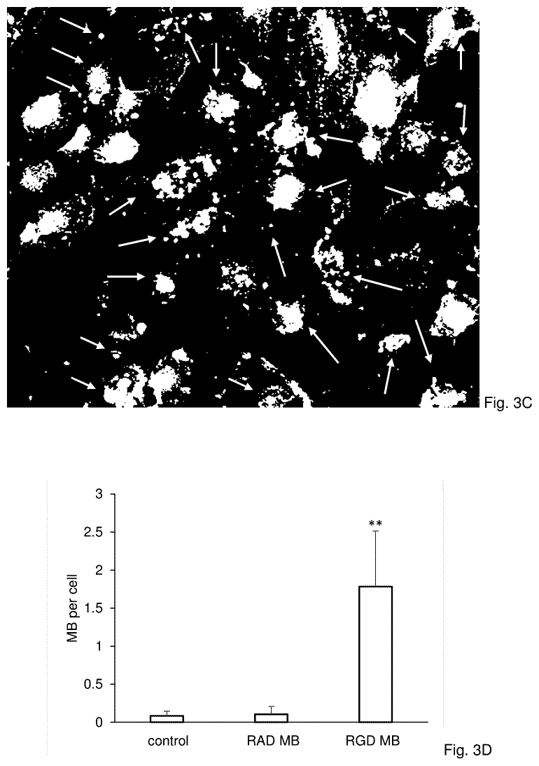

The targeting efficiency of cRGD-modified PBCA MBs (see FIG. 3C) on TNF-alpha activated human umbilical vein endothelial cells (HUVEC) was compared with cRAD-modified PBCA MBs (see FIG. 3B) and unmodified PBCA MBs (see FIG. 3A) using in vitro flow chamber assays. Endothelial cells from human umbilical vein (HUVEC, human umbilical vein endothelial cell) were activated with TNF-alpha for 4 hours before the activated HUVECs were exposed to 107 MBs in the flow chamber. cRAD-modified PBCA MBs prepared under the same conditions served as a negative control for cRGD-modified PBCA MBs. The MBs were stained with rhodamine B. Hoechst and WGA 488 were used to stain the nuclei and cell membrane, respectively. The cRGD-modified PBCA MBs were found to have a significantly higher affinity for activated HUVECs. In particular, a target efficiency of 0.22±0.09 per HUVEC was achieved (see FIG. 3D).

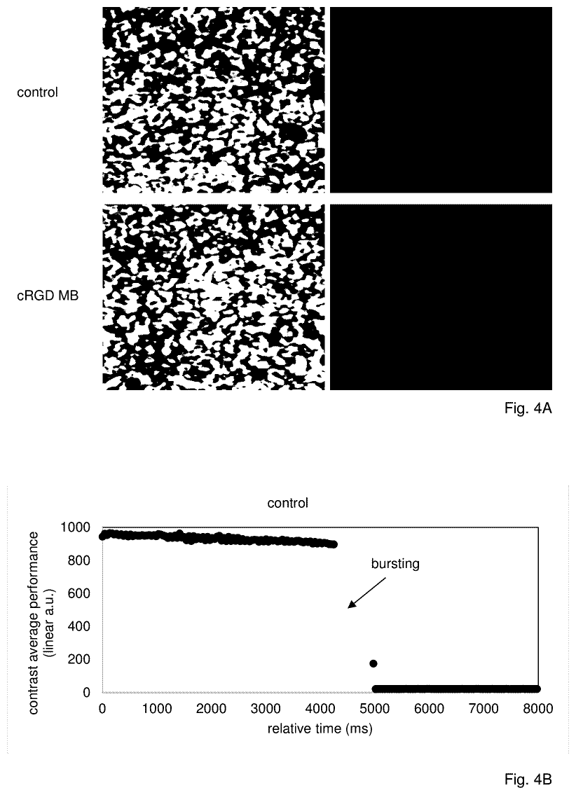

Echogenicity was assessed using the Vevo 3100 ultrasound device. For this, 3×10⁵ MBs were suspended in 4.5 ml of 2% gelatin solution, and the mixture was embedded in 10% gelatin solution. US imaging was performed in nonlinear contrast mode at a center frequency of 18 MHZ, initially at a power of 4% (mechanical index 0.2), followed by a power of 100% (mechanical index 0.9) to destroy the MBs in the gelatin phantom. The quantitative analysis shows that cRGD-modified PBCA MBs and unmodified PBCA MBs exhibited similar contrast signals, each with a high and stable amplification of the monodisperse contrast signal without being significantly altered by the modification (see FIG. 4). The acoustic behavior of the MBs modified according to the invention is comparable to the echogenicity achieved by the streptavidin method.

1. A method for functionalizing a cyanoacrylate-based material comprising the following steps:
a) preparation of a cyanoacrylate-based material with ester groups;
b) provision of a ligand with an amino group;
c) contacting the cyanoacrylate-based material with the ligand under conditions favoring aminolysis of the amino group of the ligand and the ester groups of the cyanoacrylate-based material; and optionally:
d) obtaining the cyanoacrylate-based material functionalized with the ligand.

2. The method according to claim 1, wherein the provided cyanoacrylate-based material comprises one or more polymers
and/or
the cyanoacrylate-based material is provided in the form of particles, capsules or vesicles.

3. The method according to claim 1, wherein the amino group is a primary amino group.

4. The method according to claim 1, wherein:
the ligand is a functional compound selected from the group consisting of targeting ligands, diagnostics, therapeutics, macromolecules and nanomedicine constructs;
or
the ligand is a linker via which the functional compound is bound or can be bound in a further step or is bound in a further step.

5. The method according to claim 2, wherein:
the coupling rate is in the order of 10⁻²¹ to 10⁻¹⁵ mol ligand molecules per particle, capsule or vesicle
and/or
the yield of functionalized, intact particles, capsules or vesicles is greater than 50%.

6. The method according to claim 1, wherein:
step c) is carried out at a pH value of 7.25 to 8.75
and/or
step c) is carried out in the presence of a catalyst.

7. A cyanoacrylate-based material functionalized with a ligand, produced according to claim 1.

8. An ultrasonic contrast enhancer or ultrasonic-mediated drug delivery system comprising the cyanoacrylate-based material functionalized with a ligand of claim 7.

9. A method for diagnosing a disease associated with vascular inflammation, comprising administering to a subject in thereof an ultrasound contrast enhancer comprising echogenic vesicles functionalized with a peptide for integrin-mediated cell adhesion having a polymer shell and a gas core enclosed by the polymer shell, wherein the echogenic vesicles are produced according to the method of claim 1.

10. A method of treating a disease associated with vascular inflammation, comprising administering to a subject in need thereof an ultrasound-mediated drug delivery system comprising a drug and vesicles functionalized with integrin-mediated cell adhesion peptide and having a polymer shell and a gas core enclosed by the polymer shell, wherein the vesicles are produced according to the method of claim 1.

11. The method of claim 9, wherein the disease associated with vascular inflammation comprises inflammatory bowel disease, arteriosclerosis, arterial wall injury, or cancer.

12. The method of claim 10, wherein the disease associated with vascular inflammation comprises inflammatory bowel disease, arteriosclerosis, arterial wall injury, or cancer.

13. The method of claim 4 wherein the functional compound comprises a peptide for integrin-mediated cell adhesion.

14. The method of claim 13, wherein the peptide for integrin-mediated cell adhesion comprises an RGD peptide or an RAD peptide.

15. The method of claim 13, wherein the peptide for integrin-mediated cell adhesion comprises an RGDfK peptide or an RADfK peptide.

16. The method of claim 6, wherein step c) is carried out in the presence of lithium methanolate.

17. The method according to claim 2, wherein the cyanoacrylate-based material comprises a poly(alkyl cyanoacrylate).

18. The method according to claim 17, wherein the poly(alkyl cyanoacrylate) comprises poly(n-butyl cyanoacrylate).

19. The method according to claim 2, wherein the cyanoacrylate-based material comprises echogenic vesicles comprising a polymer shell and a gas core enclosed by the polymer shell.

20. The method according to claim 6, wherein step c) is carried out at a pH value of 7.75 to 8.25.
